(19) United States
(12) Patent Application Publication
(10) Pub. No.: US 2008/0041236 A1
(43) Pub. Date: Feb. 21, 2008

(54) Device for Preparing Food for Young Children

(76) Inventors: David Raouf, Vevey (CH); Mathias Och, Montreux (CH); David Veenhuys, Les Paccots (CH)

Correspondence Address: WINSTEAD PC, P.O. BOX 50784, DALLAS, TX, 75201, US

(21) Appl. No.: 11/629,451

(22) PCT Filed: Feb. 3, 2005
(86) PCT No.: PCT/CH05/00058

§ 371 Date: Feb. 9, 2007

(30) Foreign Application Priority Data
Jun. 14, 2004 (CH) 999/04

(51) Int. Cl. A47J 31/40; A61L 2/07

(52) U.S. Cl. 99/323.3

(57) ABSTRACT

A device for the preparation of powdered baby food having the capability to fill a vessel such as a baby bottle with a pre-determined amount of baby food, and also having a steam chamber to be used for sterilizing the vessels and other items. The device can be modified to allow for simultaneous parallel preparation and dispensing of multiple servings of powdered baby food.

### BACKGROUND OF THE INVENTION

1. Technical Field

The invention pertains to a device for preparing baby food from powder.

2. History of Related Art

Devices for preparing baby food from powder usually consist of a water reservoir, in which a quantity of water is heated to a desired temperature and maintained at this temperature by means of a thermostat. For example, a gravity-type activated carbon filter for removing contaminants from the water may be arranged upstream of the water reservoir.

An apparatus of this type is disclosed in WO 99/15056. In this case, a pre-filtered water volume is disinfected, for example, with UV light and then heated to and maintained at an adjustable temperature such that correctly tempered water for the immediate preparation of powdered baby food is available at all times. The device prevents the user and the baby from being scalded by excessively hot water.

If the heated water is not used for an extended period of time, it is possible that residual germs multiply under the ideal temperatures and thusly contaminate the baby food.

### SUMMARY OF THE INVENTION

The invention is based on an objective of making devices for preparing baby food faster, safer and more user-friendly, wherein the food preparation as well as the preparation of the drinking vessel should be largely automated and the sterility of the food should be ensured in any case.

### BRIEF DESCRIPTION OF THE DRAWINGS

A more complete understanding of various embodiments of the Device for Preparing Baby Food of the present invention may be obtained by reference to the following Detailed Description, when taken in conjunction with the accompanying Drawings, wherein:

FIG. 1 illustrates a schematic representation of a first embodiment of a device for preparing baby food from powder.

FIG. 2 illustrates a schematic representation of a second embodiment of a device for preparing baby food from powder.

### DETAILED DESCRIPTION OF THE DRAWINGS

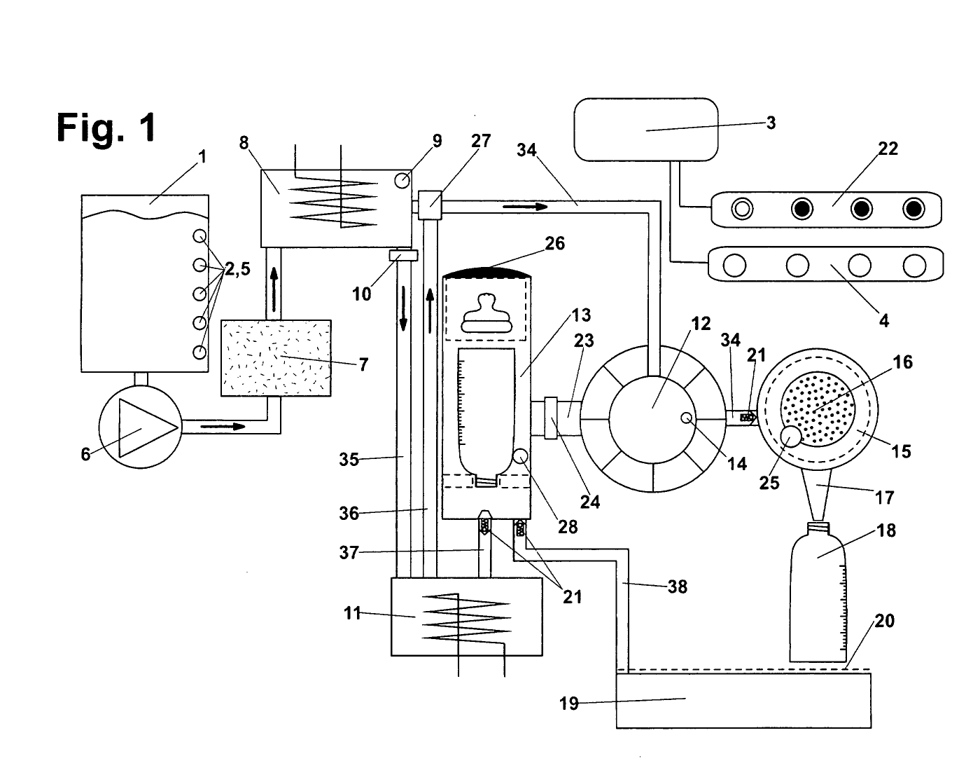

FIG. 1 schematically shows a first embodiment of the invention. A water reservoir 1 can be filled with tap water. The scope of the invention also includes alternative embodiments, in which the fresh water supply is realized in the form of a direct connection to the drinking water supply. A water reservoir 1 is not required in such embodiments. The water reservoir 1 is equipped with a water level sensor 2 that transmits corresponding information to an electronic control system 3 once a minimum water level is reached in the water reservoir 1, the control system 3 subsequently outputs an acoustical and/or optical warning signal on a display unit 4 and stops the operation of the device until the water is refilled. The water level sensor 2 may also be composed, for example, of several water level sensors 2 such that the control system 3 can output the current filling level on the display unit 4 based on the signals of the water level sensors. Several zero-dimensional water level sensors 2 may collectively form a one-dimensional sensor unit 5. It would also be conceivable to use a single sensor unit 5 that continuously measures the filling level. In addition, the water reservoir 1 may completely or partially consist of transparent material or a float may be provided for visually checking the water level in the water reservoir 1 and reading out the water level, for example, on a scale. A water pipe arranged on the bottom of the water reservoir 1 serves for feeding water to a water pump 6. The water pump 6 pumps the water to a heating unit 8 through a filter unit 7. The filter unit 7 operates, for example, with activated carbon or in accordance with the reverse-osmosis principle and may simply be realized in the form of an exchangeable water filter. The filtered water is conveyed from the filter unit 7 to the heating unit 8. This heating unit 8 may consist, for example, of an electric continuous-flow heater that heats the water to a temperature that lies near but below the boiling point, for example, to a temperature between 90° C. and 100° C. This heating process kills any pathogenic germs that may still be present in the water despite the filtration and additionally disinfects the water.

The water temperature of the water being discharged from the heating unit 8 is measured with the aid of a temperature sensor 9 and transmitted to the control system 3. The electric lines leading from and to the control system 3 are not illustrated in the figures in order to provide a better overview. Each component that is controlled by the control system 3 or transmits measuring data to the control system is connected thereto via at least one electric line. This applies, among other things, to all sensors, motors, solenoid valves and heating elements. The water flow rate, i.e., the delivery rate of the water pump 6, and the heating power of the heating unit 8 can be regulated based on the data of the temperature sensor 9 such that the water is discharged from the heating unit 8 with a constant temperature T1. The water being discharged from the heating unit 8 can either be conveyed to a steam generating unit 11 via a water pipe 35 or to a cooling unit 12 via a pipe 34 with the aid of shut-off valves 10, 27, wherein the water is subsequently delivered to a dosing unit 15. The steam generating unit 11 additionally heats the inflowing water beyond its boiling point. The thusly created steam can be used for sterilizing the device or for sterilizing, for example, the bottle and the nipple or for heating solid or semi-solid baby food in a steam chamber 13. The steam chamber 13 has an opening that can be sealed with a safety cover 26.

When preparing a beverage, the water is conveyed through a cooling unit 12 in which it is air-cooled to a drinkable temperature, for example, between 20° C. and 45° C. that can be adjusted on the control panel 22 with the aid of an adjustable electric fan, wherein the cooling unit 12 also contains an integrated temperature sensor 14, the temperature data of which can be used by the control system 3 for adjusting the fan speed of the cooling unit 12 and the delivery rate of the water pump 6. In this case, it is ensured that the temperature can never exceed 45° C. such that the baby cannot be burnt or scalded due to excessively hot beverages. The correspondingly cooled water is subsequently delivered to a dosing unit 15 via the extension of the pipe 34 and a spring-loaded pressure relief valve 21. In the dosing unit 15, the water flows through a capsule 16 containing the powdered baby food and dissolves the powdered baby food during this process, wherein the finished beverage flows into a prepared drinking vessel 18 through an outlet 17. A sensor 25 makes it possible to determine whether or not a capsule 16 is inserted into the dosing unit 15. A retainer 19 arranged underneath the outlet 17 is realized in such a way that spilled or overflowing water or beverage can be accommodated by the retainer 19. For example, the retainer 19 is covered with a screen cover 20, onto which the drinking vessel 18 can be placed. The steam chamber 13 is also connected to the retainer 19 by means of a pipe 38, wherein this pipe 38 contains a spring-loaded pressure relief valve 21 that makes it possible to discharge excess steam, when using the steam chamber 13 for sterilization or for heating purposes, and condensate from the steam chamber 13 into the retainer 19. This is the reason why the pipe 38 is essentially arranged at the lowest point of the steam chamber 13. The cooling unit 12 is connected to the steam chamber 13 by means of an air duct 23. A stopper 24, for example, in the form of a valve or a slide gate is arranged in the air duct 23 and can be manually or automatically opened after the sterilization of appliances in the steam chamber 13, and the cooling unit 12 subsequently blows cooling air into the steam chamber 13. The safety cover 26 of the steam chamber 13 is opened during this process.

In addition to a display unit 4, the inventive device also features at least one control panel 22. The control panel 22 consists, for example, of buttons, switches, controllers or other input devices for enabling the user of the device to input data and commands into the control system 3. The control panel 22 of the device makes it possible, for example, to switch the device on and off or to select the desired temperature of the beverage. The display unit 4 may consist, for example, of multi-colored light-emitting diodes and/or a liquid crystal display. However, the display unit 4 may also comprise acoustic signal generators. A person skilled in the art is familiar with other options for displaying the operating state with the aid of monitors or other visual and/or acoustic signaling devices and for visualizing and acknowledging input data. It would also be conceivable to entirely or partially combine the display unit 4 and the control panel 22 into a single unit, for example, by utilizing a touch-screen.

The control system 3 receives and processes the electric signals of all sensors and controls all actuators such as, for example, the water pump 6, the shut-off valves 10, 27, the fans and the heating elements in the heating unit 8 and the steam generating unit 11. It essentially consists of a microprocessor with a non-volatile data memory. For example, a re-writable data memory containing the control program, and a volatile random access memory for the intermediate storage of current operating parameters, for example, temperature data.

The device is multi-functional and essentially able to perform six tasks. First, the baby bottle and a nipple as well as other appliances, for example, a pacifier, can be sterilized in the steam chamber 13. In this case, the valve 27 in the pipe 34 leading to the cooling unit 15 is completely closed and the valve 10 in the water pipe 35 leading to the steam generating unit 11 is opened. The valves used consist, for example, of solenoid valves. The water pre-heated in the heating unit 8 flows from the heating unit 8 into the steam generating unit 11, in which it is additionally heated and then flows into the steam chamber 13 in the form of steam via the steam duct 37, wherein the steam chamber is sealed with a safety cover 26. Once an adjusted excess pressure is reached, the steam flows into the retainer 19 that collects the steam condensate via the spring-loaded pressure relief valve 21 and the pipe 38. The pressure relief valve 21 allows a certain overpressure in the steam chamber, for example, an overpressure of 2.4 bar and therefore a steam temperature of 125° C. required for the sterilization.

Second, the appliances can also be cooled after the sterilization in the steam chamber 13. In this case, the safety cover 26 is at least partially opened and the stopper 24 is completely opened such that the cooling unit 12 is able to blow air into the steam chamber 13 via the air duct 23 and this air can escape via the steam chamber opening.

Third, the device can be used for beverage preparation. For the actual beverage preparation, the valve 27 is opened such that the pipe 34 between the heating unit 8 and the cooling unit 12 is released. In the direction of the steam generating unit 11, in contrast, the valve 27 leading to the steam duct 36 remains closed. The hot water flows into the cooling unit 12 in which it is cooled to the desired drinking temperature and then into the dosing unit 15 in which it dissolves the powdered baby food, wherein the finished beverage is discharged into the prepared drinking vessel 18 through the funnel-shaped outlet 17.

Fourth, after the beverage preparation, the pipe 34, the cooling unit 12, the dosing unit 15 and the outlet 17 can be rinsed with hot or cold water, wherein the adjustment of the valve 27 remains unchanged. The rinsing water is discharged into the retainer 19 through the outlet 17.

Fifth, after the device has been rinsed, the pipe 34 and the cooling unit 12 can be sterilized with steam. For this purpose, the valve 27 is opened such that the steam can flow from the steam generating unit 11 in the direction of the cooling unit 12 via the steam duct 36 and the pipe 34. The pipe between the valve 27 and the heating unit 8 is closed, in contrast, with the aid of the valve 27. In addition, the valve 10 is opened and the steam generating unit 11 is active and generates steam that flows to the cooling unit 12 via the valve 27, passes through the dosing unit 15 and escapes through the outlet 17.

Sixth, solid or semi-solid food can also be heated in the steam chamber 13. The situation is identical to that described above with respect to the second task, namely the sterilization of appliances. In this case, food is placed into the steam chamber 13 through the steam chamber opening, for example, in a removable screen basket, instead of appliances to be sterilized. The steam temperature is maintained lower than during the sterilization. A temperature sensor 28 in the interior of the steam chamber 13 acquires the temperature data required for heating the food to the desired temperature.

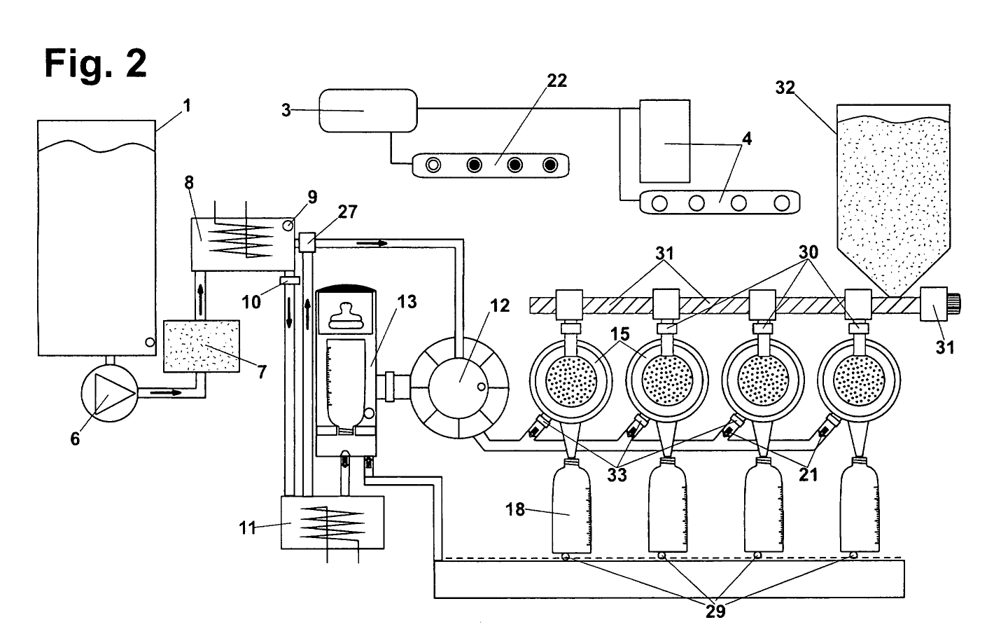

FIG. 2 schematically shows a second embodiment of a device according to the invention. This device is able to prepare several baby beverages simultaneously, i.e., in a parallel fashion. In contrast to the first embodiment, several dosing units 15 with several outlets 17 are provided. A sensor 29 may be arranged underneath each outlet in order to deliver data on the presence or absence of a drinking vessel 18 underneath the outlet 17 to the control system 3. If the sensor 29 does not detect a drinking vessel 18, the shut-off valve 30 positioned upstream of the dosing unit 15 is not opened and no beverage powder flows into the dosing unit 15. A valve 33 arranged downstream of the pressure relief valve 21 in the branch extending from the cooling unit 12 in the direction of the dosing unit 15 is closed simultaneously, i.e., in the absence of a drinking vessel 18 underneath the outlet 17. This means that the beverage is not discharged from the outlet 17, under which no drinking vessel 18 is positioned. In addition, this embodiment features an alternative design of the dosing unit 15. The powdered baby food is not transported from a powder reservoir 32 into the dosing unit 15 by means of powder-filled capsules 16, but rather a transport mechanism 31, wherein the valve 30 is closed as soon as the correct quantity of powder was introduced into the dosing unit 15. In order to detect the correct powder filling, additional sensors may be provided for detecting the quantity of powder introduced into the dosing unit 15. The transport mechanism 31 may be realized, for example, in the form of a worm gear with serial feed. In a device for the simultaneous and parallel preparation of beverages, the steam chamber may, according to the invention, have such dimensions that a number of drinking vessels 18 that corresponds to the number of outlets 17 can be simultaneously sterilized.

However, a person skilled in the art is familiar with other options for transporting and dosing the powder, for example, a parallel feed. These options also fall under the scope of the present invention although they are not described in detail. Instead of utilizing a powder reservoir 32 that can be filled with any powder, it would also be conceivable to utilize powder packages that are placed into the device directly or with the aid of an adapter such that a powder reservoir 32 can be eliminated.

Generally speaking, other possible combinations of the above-disclosed characteristics that may appear trivial to a person skilled in the art also fall under the scope of the invention. When using other beverage powders, the device naturally can also be used for preparing different hot and warm beverages, for example, coffee and tea.

1. A device for preparing baby food from powder, the device comprising:
a water pump;
a filter unit;
an outlet;
a retainer;
a heating unit;
a display unit
an operating panel;
a cooling unit fluidly coupled to the heating unit by a first pipe, the cooling unit comprising a temperature sensor and capable of cooling water heated in the heating unit and conveyed to the cooling unit via the first pipe to a temperature suitable for oral consumption by a baby, wherein a control system regulates the temperature to a predetermined adjustable value;
a steam chamber;
a steam generating unit fluidly coupled to the heating unit via a second pipe; wherein water heated in the heating unit can be conveyed to the steam generating unit through the second pipe;
a steam duct fluidly coupling the steam generating unit and the steam chamber, and through which the steam flows from the steam generating unit into the steam chamber;
a third pipe fluidly coupling the steam chamber to the retainer, wherein the third pipe extends away from a lowest point of the steam chamber;
a dosing unit in which the powder is combined with the water that was heated in the heating unit and subsequently cooled in the cooling unit in order to produce a beverage that flows into a prepared vessel through an outlet in the dosing unit; and
an electronic control system.

2. The device for preparing baby food from powder according to claim 1, wherein the heating unit heats the water to a temperature between 90° C. and 100° C., and the temperature of the beverage lies between 20° C. and 45° C. after it passes through the cooling unit.

3. The device for preparing baby food from powder according to claim 1, further comprising a plurality of pressure relief valves capable of generating an overpressure in the steam chamber and the first and second pipes.

4. The device for preparing baby food from powder according to claim 1, further comprising:
a plurality of shut-off valves capable of directly conveying the heated water from the heating unit to the cooling unit or for directly conveying the heated water from the heating unit into the steam generating unit, wherein the steam can be subsequently conveyed either to the steam chamber via a steam duct or to the cooling unit via a steam duct.

5. The device for preparing baby food from powder according to claim 1, wherein:
the steam chamber is sealed in a steam-tight fashion with a safety cover and the steam chamber is connected to the retainer via a fourth pipe having one of the plurality of pressure relief valves disposed therein.

6. The device for preparing baby food from powder according to claim 1, wherein:
the cooling unit comprises a fan-assisted air cooling unit.

7. The device for preparing baby food from powder according to claim 6, further comprising:
an air duct having a stopper, wherein the stopper can be opened in order to cool an appliance sterilized in the steam chamber, wherein the fan of the cooling unit blows ambient air into the steam chamber.

8. The device for preparing baby food from powder according to claim 1, further comprising:
means for introducing a receptacle containing a solid baby food into the steam chamber in order to heat the solid baby food with the aid of steam.

9. The device for preparing baby food from powder according to claim 1, further comprising:
means for detecting and displaying a water level in a water reservoir.

10. The device for preparing baby food from powder according to claim 1, further comprising:
means for parallel and simultaneous preparation of a plurality of beverages.

11. The device for preparing baby food from powder according to claim 10, further comprising:
a plurality of sensors for detecting the presence of a drinking vessel underneath the outlet in the dosing unit, and a plurality of valves that make it possible to separately control a supply of powder and water to each individual dosing unit.

12. The device for preparing baby food from powder according to claim 1, wherein
the powdered baby food is delivered to the dosing unit by means of a plurality of powder capsules, and a sensor detects the presence of one of the plurality of powder capsules.

13. The device for preparing baby food from powder according to claim 1, further comprising:
a powder receptacle, from which the powdered baby food can be delivered to the dosing unit by means of a transport mechanism.
